(19) United States
(12) Patent Application Publication
(10) Pub. No.: US 2026/0263780 A1
(43) Pub. Date: Sep. 10, 2026

(54) BLOOD PUMP WITH MODIFIED LINER

(71) Applicant: Abiomed Europe GmbH, Aachen (DE)

(72) Inventor: Tino Andreas BOELKE, Aachen (DE)

(73) Assignee: Abiomed Europe GmbH, Aachen (DE)

(21) Appl. No.: 19/666,923

(22) Filed: May 4, 2026

Related U.S. Application Data
(62) Division of application No. 18/381,233, filed on Oct. 18, 2023.
(60) Provisional application No. 63/417,029, filed on Oct. 18, 2022.

(51) Int. Cl. A61M 60/81; A61M 60/13; A61M 60/237; A61M 60/806; A61M 60/857; F04D 3/02; F04D 7/00; F04D 29/18

(52) U.S. Cl. CPC A61M 60/81; A61M 60/13; A61M 60/237; A61M 60/806; A61M 60/857; F04D 3/02; F04D 7/00; F04D 29/181; A61M 2205/0238; A61M 2205/0266

(57) ABSTRACT

A blood pump is disclosed herein, that may include one or more struts configured to be coupled to a catheter, the strut(s) defining a housing having a blood inlet and a blood outlet, and having an inner surface and an outer surface opposite the inner surface. The blood pump may include an inner coating disposed on the inner surface at the blood inlet and extending axially towards the blood outlet, and may include an outflow tube coupled to the outer surface, the outflow tube surrounding the blood outlet and surrounding at least a first portion of the inner coating, and extending axially beyond the blood outlet. The blood pump may include an impeller disposed within the housing, the impeller including at least one blade. Preferably, the inner coating has an axial length configured to surround 20% or less of the total length of the impeller.

### CROSS-REFERENCE TO RELATED APPLICATIONS

The present disclosure is a divisional of U.S. Ser. No. 18/381,233, filed Oct. 18, 2023, and also claims priority to U.S. Provisional Patent Application No. 63/417,029, filed Oct. 18, 2022, the contents of which are each incorporated by reference herein in its entirety.

### TECHNICAL FIELD

The present disclosure is drawn to blood pumps, and specifically to blood pumps with modified liners to accommodate specific impellers.

### BACKGROUND

Conventional blood pumps use an impeller within a housing to move blood within a patient's body. To be effective in assisting the patient, the blood pump must meet certain performance characteristics. Typically, an impeller with relatively long impeller is used to satisfy some of those characteristics, as shortening the rotor yields reduces hydraulic output. However, as the need for smaller blood pumps grows, a way to improve hydraulic output with smaller rotors will be required.

### BRIEF SUMMARY

In various aspects, a blood pump may be provided that may include one or more struts (which may be, e.g., nitinol) configured to be coupled to a catheter. The one or more struts may define a housing having a blood inlet and a blood outlet, the one or more struts having an inner surface and an outer surface opposite the inner surface. The blood pump may include an inner coating (which may be, e.g., a polyurethane) disposed on the inner surface at the blood inlet and extending towards the blood outlet. The blood pump may include an outflow tube coupled to the outer surface. The outflow tube may surround the blood outlet and may surround at least a first portion of the inner coating. The outflow tube may extend axially beyond the blood outlet. The blood pump may include an impeller disposed within the housing, the impeller including at least one blade. In some embodiments, a wrap angle of the at least one blade relative to a central axis may be at least 100 degrees, at least 180 degrees, and/or at least 200 degrees.

In some embodiments, a leading edge angle of the at least one blade relative to a central axis may be equal to the trailing edge angle of the at least one blade relative to the central axis. In some embodiments, the leading edge angle and the trailing edge angle are 55-60 degrees. In some embodiments, the at least one blade has an axial length that is 7.5 mm or less, 7 mm or less, and/or 6.5 mm or less.

In some embodiments, the blood pump may include an outer coating disposed on the outer surface of the one or more struts and coupled to the outflow tube. The outer coating may be disposed at the blood inlet and extend no further than a trailing end of the inner coating. In some embodiments, the blood pump may be free of an outer coating disposed on the outer surface of the one or more struts.

In some embodiments, the inner coating may have an axial length configured to surround a length of the at least one blade, the length being 20% or less of a total length of the impeller.

In some embodiments, the inner coating has an axial length configured to surround a length of the impeller, the length being 20% or less of a total length of the impeller, 15% or less of a total length of the impeller, and/or 10% or less of a total length of the impeller.

In some embodiments, a leading edge of the at least one blade is not surrounded by the inner coating and may be configured to be positioned a fixed distance from a trailing end of the inner coating.

In various aspects, a blood pump may be provided. The blood pump may include one or more struts coupled to a catheter, where the one or more struts may define a housing having a blood inlet and a blood outlet, the one or more struts having an inner surface and an outer surface opposite the inner surface. The blood pump may include an inner coating disposed on the inner surface at the blood inlet and extending towards the blood outlet. The blood pump may include an outflow tube coupled to the outer surface, where the outflow tube may surround the blood outlet, may surround at least a first portion of the inner coating, and may extend axially beyond the blood outlet. The blood pump may include an impeller disposed within the housing, the impeller including at least one blade.

In various aspects, a blood pump may be provided. The blood pump may include a pump housing coupled to a catheter configured for insertion into a blood vessel. The pump housing may include an inner layer disposed on an inner surface of an expandable mesh layer defining a blood inlet and a blood outlet. The inner layer may have a first end at the blood inlet and a second end offset axially from the first end by a first predetermined distance (D1). The blood pump may include an impeller disposed within the pump housing, the impeller including at least one blade. The impeller may have a first end and a second end, where the first end of the impeller may be offset axially from the first end of the inner layer by a second predetermined distance (D2). The second end of the impeller may be offset axially from the first end of the inner layer by a third predetermined distance (D3). D3 may be greater than D2, and either D2≥D1, or D1−D2≤20% of an axial length of the impeller.

In various aspects, a system may be provided, that may include a blood pump as disclosed herein, and a controller operably coupled to the blood pump.

In various aspects, a kit may be provided, that may include a blood pump as disclosed herein, and a controller configured to be operably coupled to the blood pump.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a portion of a blood pump.

FIG. 2 is an illustration of a portion of a blood pump.

FIG. 3A is an illustration of a top view of an impeller.

FIG. 3B is an illustration of an end view of an impeller.

FIG. 4A is a graph showing flow rates at different pressure heads for an embodiment of a blood pump vs. a reference blood pump.

FIG. 4B is a graph showing hydraulic efficiencies at different flow rates for an embodiment of a blood pump vs. a reference blood pump.

FIG. 5 is a schematic illustration of the use of a blood pump.

FIG. 6A-6E are cross-sectional schematics showing different arrangements and combinations of inner coating, struts, outer coating, and outflow layers.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Blood pumps often include an impeller near an end of a catheter, allowing the impeller to be placed within the body of a patient and move blood from one location to another. Blood pumps typically have some form of enclosure around the impeller, to impact the flow of blood generated by the impeller.

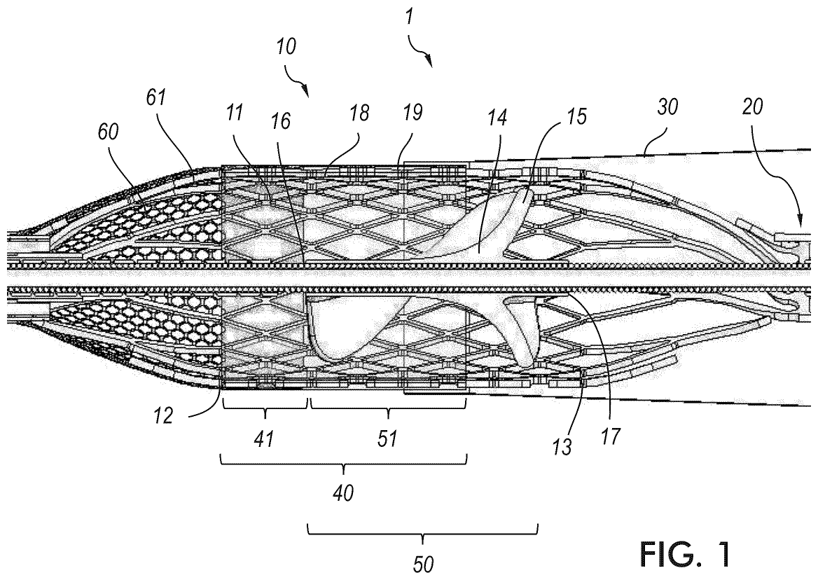

Referring to FIG. 1, an embodiment of a blood pump may be provided. The blood pump 1 includes a housing 10 coupled to a catheter 20. Typically, a distal end of a catheter will be coupled to a proximal end of a housing, although other configurations are possible.

The housing includes a series of struts 11, within which an impeller 14 with at least one blade 15 is housed. The rotation of the impeller around a central axis causes blood to flow, e.g., from a blood inlet 12 to a blood outlet 13. The housing includes an inner coating layer 18 and an outer coating layer 19 around the struts. The inner coating may define an inner surface. The inner surface may be a smooth surface. As used herein, the term “smooth surface” is intended to refer to a surface that is free of protrusions, cavities, dimples, vents, or other such deviations that extend from (or into) the surface by more than a certain distance. The distance is preferably no more than 0.5 mm, more preferably no more than 0.25 mm, and still more preferably no more than 0.1 mm.

The coating may extend from the blood inlet towards the blood outlet by a fixed distance 40. An outflow tube 30 may be coupled to the outer coating layer. The outflow tube may cover and surround the blood outlet. The impeller may be offset from the blood inlet by a predetermined distance 41. The impeller may be positioned such that the leading edge 16 of the impeller is surrounded by the coating layers. The trailing edge 17 of the impeller may also be surrounded by the coating, or may extend beyond the trailing end of the coating. However, if the impeller extends beyond the coating, at least a portion 51 of the entire length 50 of the impeller is surrounded by the coating layers. In some embodiments, that portion 51 is at least 60% of the length of the impeller (i.e., axial length of portion 51≥60%×the length of impeller 50). In some embodiments, that portion 51 is 50-60% of the length of the impeller (i.e., 50%×the length of impeller 50≤axial length of portion 51≤60%×the length of impeller 50).

In some embodiments, a mesh 60 may be included. The mesh 60 may define smaller openings than the openings defined by the struts forming the housing. In some embodiments, the mesh may be directly coupled to the struts forming the housing. In some embodiments, the mesh may be directly coupled to one or more struts 61 that are upstream (in the direction of the flow of blood) from the struts forming the housing. In some embodiments, the mesh may not be directly coupled to any struts. The mesh may be positioned upstream from the impeller. In some embodiments, the mesh may be positioned within an interior volume of space defined by the struts. In some embodiments, the mesh may be positioned external to the struts.

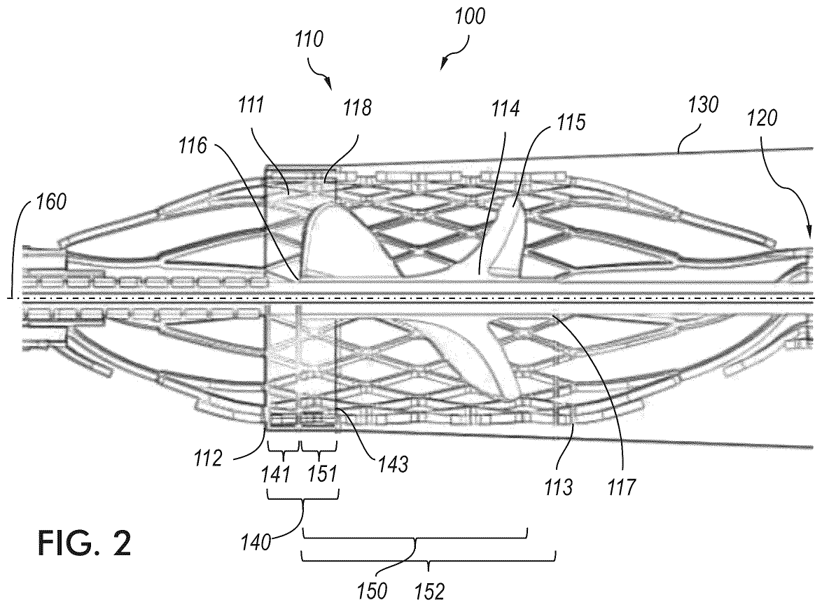

Referring to FIG. 2, in some embodiments, the blood pump 100 may include one or more struts 111. In some embodiments, the struts may form an expandable mesh layer. The strut(s) may be any appropriate material, such as nitinol. In some embodiments, the strut(s) may be configured to be coupled to a catheter 120. In some embodiments, housing may be coupled to a catheter configured for insertion into a blood vessel.

The one or more struts may define a housing 110 having a blood inlet 112 and a blood outlet 113. Blood is configured to flow from the blood inlet, past or around the impeller, and out through the blood outlet. The one or more strut(s) may have an inner surface and an outer surface opposite the inner surface. The inner surface is configured to face towards a central axis 160, the outer surface configured to face away from the central axis.

The blood pump may include an inner coating 118. The inner coating may be a single layer of a coating disposed on an inner surface of the struts.

The inner coating may be any appropriate coating material, such as a polyurethane. The inner coating may be disposed on the inner surface at the blood inlet and extending axially partially towards the blood outlet for a fixed axial distance 140 that is less than an axial length of the housing. That is, the inner coating may not extend the entire axial distance between the blood inlet and the blood outlet.

The blood pump may include an outflow tube 130 coupled to the outer surface. The outflow tube may be external to and surround the blood outlet and may surround at least a first portion of the inner coating. In FIG. 2, the outflow tube is shown as surround the entire fixed axial distance 140 that defines the axial length of the inner coating, but in other embodiments, the outflow tube does not extend axially to the blood inlet. The outflow tube may extend axially beyond the blood outlet.

The outflow tube is preferably collapsible. The outflow tube may be composed of any suitable biocompatible material, such as a suitable polymer, such as polyurethane, polyamide, nylon, or silicone. In some embodiments, the outflow tube may be polytetrafluoroethylene (PTFE).

The blood pump may include an impeller 114 disposed within the housing. The impeller may include at least one blade 115. In some embodiments, the impeller preferably includes two blades. In some embodiments, the impeller more preferably includes at least two blades.

In some embodiments, the leading edge of the impeller and/or the at least one blade is offset from the blood inlet by a predetermined distance 141.

In some embodiments, the at least one blade has an axial length 150 that is 7.5 mm or less. In some embodiments, the axial length is 7 mm or less. In some embodiments, the axial length if 6.5 mm or less. In some embodiments, the length is at least 3 mm. In some embodiments, the length is at least 4 mm. In some embodiments, the length is at least 3 mm. In some embodiments, the length is at least 5 mm. In some embodiments, the length is at least 3 mm. In some embodiments, the length is at least 6 mm.

In some embodiments, the inner coating may have an axial length (e.g., fixed axial distance 140) configured to surround a portion of the impeller, the portion having an axial length 151 that is 50% or less of a total axial length 152 of the impeller. In some embodiments, the inner coating may have an axial length (e.g., fixed axial distance 140) configured to surround a portion of the impeller, the portion having an axial length 151 that is 30% or less of a total axial length 152 of the impeller. In some embodiments, the inner coating may have an axial length (e.g., fixed axial distance 140) configured to surround a portion of the impeller, the portion having an axial length 151 that is 20% or less of a total axial length 152 of the impeller. In some embodiments, the portion may have an axial length that is 15% or less of a total axial length of the impeller. In some embodiments, the portion may have an axial length that is 10% or less of a total axial length of the impeller.

In some embodiments, the inner coating may have an axial length (e.g., fixed axial distance 140) configured to surround a portion of the at least one blade, the portion having an axial length 151 that is 50% or less of a total axial length 152 of the at least one blade. In some embodiments, the inner coating may have an axial length (e.g., fixed axial distance 140) configured to surround a portion of the at least one blade, the portion having an axial length 151 that is 30% or less of a total axial length 152 of the at least one blade. In some embodiments, the inner coating may have an axial length (e.g., fixed axial distance 140) configured to surround a portion of the at least one blade, the portion having an axial length 151 that is 20% or less of a total axial length 152 of the at least one blade. In some embodiments, the portion may have an axial length that is 15% or less of a total axial length 150 of the at least one blade. In some embodiments, the portion may have an axial length that is 10% or less of a total axial length 150 of the at least one blade.

In some embodiments, the inner coating does not surround at least a portion of the at least one blade. In some embodiments, a leading edge 116 of the at least one blade is not surrounding by the inner coating. In some embodiments, the inner coating does not surround at least a portion of the impeller. In some embodiments, there is an axial gap (not shown) or axial separation between the trailing end 143 of the inner coating and the leading edge of the at least one blade.

In some embodiment, the leading edge of the at least one blade may be a fixed distance from a trailing end of the inner coating. In some embodiments, the trailing end of the inner coating is, axially, between the leading edge of the at least one blade and the trailing edge of the at least one blade. In some embodiments, the trailing end of the inner coating is, axially, between the leading end of the inner coating and the leading edge of the at least one blade.

In some embodiments, the inner coating may have a first end at the blood inlet and a second end offset axially from the first end by a first predetermined distance (D1) (e.g., fixed axial distance 140). In some embodiments, the impeller may have a first end and a second end, where the first end of the impeller may be offset axially from the first end of the inner layer by a second predetermined distance (D2) (e.g., predetermined distance 141). The second end of the impeller may be offset axially from the first end of the inner layer by a third predetermined distance (D3) (e.g., the sum of predetermined distance 141 and the total axial length 152 of the impeller). In various embodiments, D3 may be greater than D2, and either D2≥D1 or D1−D2≤20% of an axial length of the impeller.

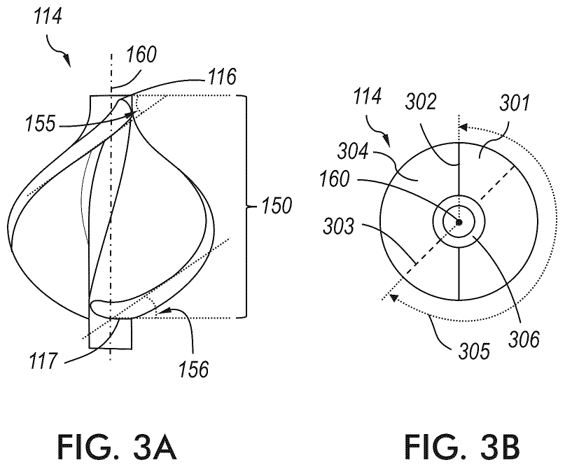

Referring to FIG. 3A, the impeller is configured to rotate around a central axis 160. In some embodiments, the impeller blade(s) may have a leading edge angle 155 and a trailing edge angle 156, relative to the central axis. The leading edge angle and trailing edge angle are the angles formed by a line normal to the axis and a line parallel to the blade's surface at the outer edge of the blade at the leading edge or trailing edge, respectively.

In some embodiments, a leading edge angle of the at least one blade relative to a central axis may be equal to the trailing edge angle of the at least one blade relative to the central axis.

In some embodiments, a leading edge angle of the at least one blade relative to a central axis may be different from the trailing edge angle of the at least one blade relative to the central axis. In some embodiments, the leading edge angle and/or the trailing edge angle may be 45-60 degrees. In some embodiments, the leading edge angle and/or the trailing edge angle may be 55-60 degrees. In some embodiments, the leading edge angle and/or the trailing edge angle may be 50-55 degrees. In some embodiments, the leading edge angle and/or the trailing edge angle may be 45-50 degrees. The smaller the angle, the higher the pitch of the blades.

Referring to FIG. 3B, an impeller can be seen looking from the leading edge down toward the trailing end of the impeller. A first blade 301 is shown, with a leading edge 302 and a trailing edge 303. The trailing edge is shown as a dashed line, as it is positioned behind the leading portion of second blade 304. The wrap angle 305 of the first blade is shown. The wrap angle is the angle formed by the blade indicating the amount the blade “wraps around” the central axis in a helical pattern. So, a blade that is straight, positioned parallel to the central axis, will have a wrap angle of 0°, while a blade that forms one complete helix turn will have a wrap angle of 360°. The wrap angle is generally considered to be the angle between (1) an imaginary line from the central axis, through the leading edge at the point the leading edge of the first blade attaches to a central hub 306, and (2) an imaginary line from the central axis, through the trailing edge at the point the trailing edge attaches to the central hub. In some embodiments, a wrap angle of the at least one blade relative to a central axis may be at least 100 degrees. In some embodiments, a wrap angle of the at least one blade relative to a central axis may be at least 180 degrees. In some embodiments, a wrap angle of the at least one blade relative to a central axis may be at least 200 degrees.

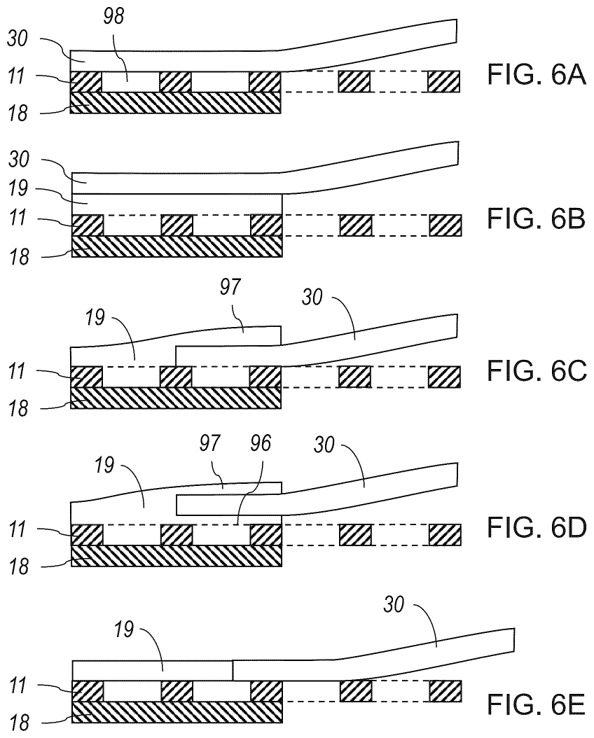

Referring to FIG. 6A, in some embodiments, the blood pump may have the inner coating 18 disposed on an inner surface of the struts 11, and the outflow tube 30 disposed on an outer surface of the struts.

In some embodiments, as seen in FIG. 1, the blood pump may include an outer coating. The configuration of the outer coating, the inner coating, the struts, and the outflow tubing may vary. In some embodiments, the outer coating may be coupled to the outflow tube in a variety of ways. Referring to FIG. 6B, in some embodiments, the outer coating 19 may be disposed on the outer surface of the one or more struts, between the struts and the outflow tube. The outflow tube may be disposed around the outer coating. Referring to FIG. 6C, in some embodiments, the outflow tube and the outer coating are both disposed on an outer surface of the struts. In some embodiments, an inner surface of a first portion 97 of the outer coating may be in contact with an outer surface of the outflow tube, such as a portion of the outflow tube disposed on the outer surface of the struts. As will be understood, this can be reversed-a portion of an inner surface the outflow tube may be in contact with an outer surface of the outer coating. Referring to FIG. 6D, in some embodiments, an end of the outflow tube may be sandwiched between an inner surface of a first portion 97 and an outer surface of a second portion 96 of the outer coating. Referring to FIG. 6E, in some embodiments, the trailing end of the outer coating may abut a leading end of the outflow tube.

In some embodiments, the outer coating may be disposed at the blood inlet and extend axially no further than a trailing end of the inner coating. In some embodiments, at least a portion of the outer coating may be between the one or more struts and the outflow tube. In some embodiments, the blood pump may be free of an outer coating disposed on the outer surface of the one or more struts.

Referring to FIG. 6A, adjacent struts (axially and circumferentially) define spaces 98 between the struts (e.g., a space between a distal-facing surface of a first strut and a proximal-facing surface of an adjacent strut). In each such space, the volume of space may be filled with the material forming the inner coating, the material forming the outflow tube, and/or the material forming the outer coating.

The outer coating may be any appropriate coating material, such as polyurethane or polytetrafluoroethylene (PTFE).

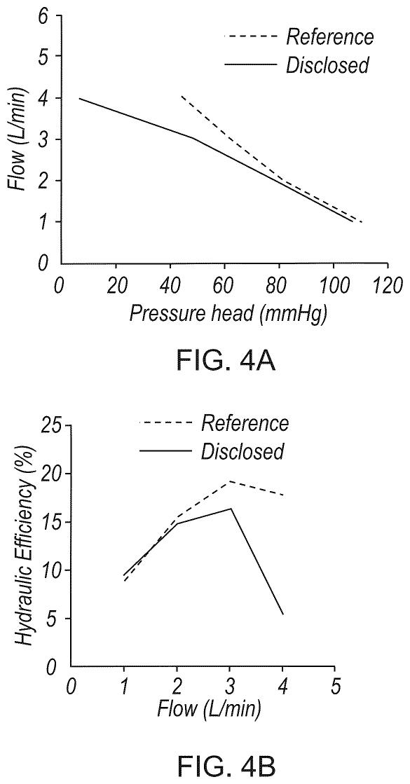

It is well known that reducing the length of the rotor blades will reduce hydraulic output. Referring to FIGS. 4A and 4B, comparisons between a commercially available blood pump, having a relatively long impeller and inner coating, and an embodiment of the present blood pump, with a shorter impeller and inner coating, can be seen, operating at the same RPM. As seen, while there is a reduction in hydraulic efficiency, the disclosed blood pump has surprisingly similar flow rate to the commercially available pump, at higher pressures.

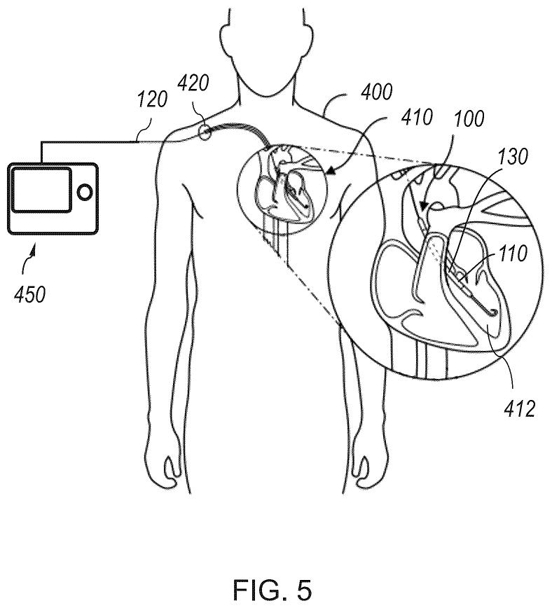

Referring to FIG. 5, in some embodiments, a system may be provided. A blood pump 100 may be inserted, at an insertion point 420, into a patient 400, the blood pump being inserted until it reaches a desired location. In FIG. 5, the device is shown as being inserted where the blood inlet 112 is positioned in the left ventricle 412 of the patient's heart 410. The blood will flow from the left ventricle, through the blood inlet, the housing 110, and out through the outflow tube 130 and into the aorta. A controller 450 may be used to control the blood pump. The blood pump may be operably coupled to the controller via, e.g., the catheter 120. The blood pump may be removably coupled from the controller.

In some embodiments, a kit may be provided. The kit may include a blood pump as disclosed herein, and a controller configured to be operably coupled to the blood pump.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A system comprising:
a blood pump, comprising:
one or more struts configured to be coupled to a catheter, the one or more struts defining a housing having a blood inlet and a blood outlet, the one or more struts having an inner surface and an outer surface opposite the inner surface;
an inner coating disposed on the inner surface at the blood inlet and extending partially towards the blood outlet in an axial direction, the inner coating defining a smooth inner surface;
an outflow tube coupled to the outer surface, the outflow tube surrounding the blood outlet and surrounding at least a first portion of the inner coating, and extending axially beyond the blood outlet; and
an impeller disposed within the housing, the impeller including at least one blade;
wherein a wrap angle of the at least one blade relative to a central axis is at least 100 degrees; and
a controller operably coupled to the blood pump.

2. The system of claim 1, wherein the wrap angle is at least 180 degrees.

3. The blood pump of claim 1, wherein a leading edge angle of the at least one blade relative to a central axis is equal to a trailing edge angle of the at least one blade relative to the central axis.

4. The blood pump of claim 3, wherein the leading edge angle and the trailing edge angle are 55-60 degrees.

5. The blood pump of claim 1, wherein the at least one blade has an axial length that is 7.5 mm or less.

6. The blood pump of claim 1, further comprising an outer coating disposed on the outer surface of the one or more struts and coupled to the outflow tube, the outer coating disposed at the blood inlet and extending no further than a trailing end of the inner coating.

7. The blood pump of claim 1, wherein the blood pump is free of an outer coating disposed on the outer surface of the one or more struts.

8. The blood pump of claim 1, wherein the inner coating has an axial length configured to surround a length of the at least one blade, the axial length being 50% or less of a total length of the at least one blade.

9. The blood pump of claim 1, wherein the inner coating has an axial length configured to surround a length of the impeller, the axial length being 50% or less of a total length of the impeller.

10. The blood pump of claim 9, wherein the length of the impeller that is surrounded by the inner coating is 15% or less of the total length of the impeller.

11. The blood pump of claim 1, wherein a leading edge of the at least one blade is not surrounded by the inner coating and is configured to be positioned a fixed distance from a trailing end of the inner coating.

12. The blood pump of claim 1, wherein the one or more struts comprises nitinol.

13. The blood pump of claim 1, wherein the inner coating is polyurethane.

14. The blood pump of claim 1, wherein the at least one blade comprises at least two blades.

15. The blood pump of claim 1, wherein the at least one blade consists of two blades.

16. A system comprising:
a blood pump, comprising:
one or more struts coupled to a catheter, the one or more struts defining a housing having a blood inlet and a blood outlet, the one or more struts having an inner surface and an outer surface opposite the inner surface;
an inner coating disposed on the inner surface at the blood inlet extending axially partially towards the blood outlet;
an outflow tube coupled to the outer surface, the outflow tube surrounding the blood outlet and surrounding at least a first portion of the inner coating, and extending axially beyond the blood outlet; and
an impeller disposed within the housing, the impeller including at least one blade, wherein a wrap angle of the at least one blade relative to a central axis is at least 100 degrees; and
a controller operably coupled to the blood pump.

17. A system comprising:
a blood pump, comprising:
a pump housing coupled to a catheter configured for insertion into a blood vessel, the pump housing comprising an inner layer disposed on an inner surface of an expandable mesh layer defining a blood inlet and a blood outlet, the inner layer having a first end at the blood inlet and a second end offset axially from the first end by a first predetermined distance (D1); and
an impeller disposed within the pump housing, the impeller including at least one blade, the impeller having a first end and a second end, where the first end of the impeller being offset axially from the first end of the inner layer by a second predetermined distance (D2), the second end of the impeller being offset axially from the first end of the inner layer by a third predetermined distance (D3);
wherein D3>D2 and wherein either D2≥D1 or D1−D2≤50% of an axial length of the impeller; and
a controller operably coupled to the blood pump.

18. The blood pump of claim 17, wherein D1−D2≤30% of an axial length of the impeller.

19. The blood pump of claim 18, wherein D1−D2≤20% of an axial length of the impeller.
